(19) United States
(12) Patent Application Publication
(10) Pub. No.: US 2023/0241267 A1
(43) Pub. Date: Aug. 3, 2023

(54) SYSTEMS AND METHODS FOR AN AUTOMATED STERILIZATION SYSTEM

(71) Applicant: Epic Universal Technologies, LLC., Hickory, NC (US)

(72) Inventor: George Erik McMillan, Hickory, NC (US)

(21) Appl. No.: 18/105,598

(22) Filed: Feb. 3, 2023

Related U.S. Application Data
(60) Provisional application No. 63/306,252, filed on Feb. 3, 2022.

(51) Int. Cl. A61L 2/10; A61L 2/24; A61L 2/26

(52) U.S. Cl. CPC A61L 2/10; A61L 2/24; A61L 2/26; A61L 2202/14

(57) ABSTRACT

An automated UV sterilization system is provided. In some embodiments, the automated UV sterilization system includes a first UV sterilization unit including a first UV lamp, the first UV sterilization unit configured to provide UV exposure. The automated UV sterilization system includes a first sensor coupled to the first UV sterilization unit, the first sensor configured to measure the UV exposure. The automated UV sterilization system is configured to: determine dimensions of a target area surrounding UV sterilization units, determine the UV exposure to be provided based on the determined dimensions, activate the UV exposure to be provided by UV sterilization units, determine a current UV exposure within the target area, and determine whether the current UV exposure meets a target criteria to complete sterilization for the target area surrounding UV sterilization units.

### CROSS-REFERENCE TO RELATED APPLICATION

This application claims the benefit of U.S. Provisional Pat. Application No. 63/306,252, titled “UV STERILIZER TOWER AND FULLY AUTOMATIC CONTROL SYSTEM,” which was filed on Feb. 03, 2022 and is incorporated herein by reference in its entirety.

FIELD OF TECHNOLOGY The present disclosure relates generally to pathogen sterilization systems and, more specifically, to an automated and/or multi-level ultraviolet (UV) sterilization systems including an ultraviolet-C (UVC) sterilization system having a plurality of sterilization mechanisms for automatically performing controlled UVC sterilization, maximizing UVC lamp lifespan, decreasing sterilization time, for sterilizing hard to reach areas, for providing operator feedback, and/or reducing tool maintenance.

### BACKGROUND

Air purification for air conditioners and/or air ventilation systems can be important to eliminate viruses, bacteria, and/or other hazardous micro-organisms from the air flowed through these systems. One such technique makes use of Ultraviolet (UV) light. UV is a form of electromagnetic radiation with wavelength between 100 nm and 400 nm, shorter than that of visible light, but longer than X-rays. UV radiation—which is divided into three bands: UVA (315 - 400 nm), UVB (280 - 315 nm), and UVC (200- 280 nm), VUV (100-200 nm) is present in sunlight, and constitutes about 10% of the total electromagnetic radiation output from the Sun. UV light interacts with matter in a variety of ways. For example, short-wave UV light (e.g., UVC light) deactivates the DNA and RNA of microorganisms like bacteria, viruses, and other pathogens, and disrupts their ability to multiply and cause diseases. Due to this effect, UVC light can be used to quickly (e.g., within minutes) sterilize objects, large surfaces, or even the air in hospitals, medical centers, food plants, office spaces, etc. Advantageously, the UVC treatment leaves no residue, and thus, the treated object or area can be immediately used after sterilization. The UVC light used in sterilization applications has a wavelength between 200 and 280 nanometers, and more preferably a wavelength of 253.7 nm.

Conventional UV sterilization products are often deployed and left alone in areas which may pose a danger to users. Furthermore, conventional UV sterilization products can lose sterilization efficiency and effectivity over time, due to the lifespan the UV lamps used. Also, conventional UV sterilization products are not configured to sterilize and/or clean hard to reach areas such as under beds, chairs, tables, etc. For example, conventional UV sterilization products can take additional time to sterilize a hard to reach area. Thus, users can often leave the conventional UV sterilization product alone unattended during the sterilization process, posing a risk to other users in case of malfunction while the conventional UV sterilization product is unattended.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY

An automated UV sterilization system is provided. In some embodiments, the automated UV sterilization system includes a first UV sterilization unit including a first UV lamp, the first UV sterilization unit configured to provide UV exposure. The automated UV sterilization system includes a first sensor coupled to the first UV sterilization unit, the first sensor configured to measure the UV exposure. The automated UV sterilization system is further configured to: determine dimensions of a target area surrounding UV sterilization units, determine the UV exposure to be provided based on the determined dimensions, activate the UV exposure to be provided by UV sterilization units, determine a current UV exposure within the target area, and determine whether the current UV exposure meets a target criteria to complete sterilization for the target area surrounding UV sterilization units

An automated UV sterilization method is disclosed. In one embodiment, the method includes determining dimensions of a target area surrounding a first UV sterilization unit of an automated UV sterilization system, the first UV sterilization unit configured to provide UV exposure. The method includes determining the UV exposure to be provided based on the determined dimensions. The method includes activating the UV sterilization system to provide UV exposure. The method includes determining a current UV exposure within the target area. The method includes determining whether the current UV exposure meets a target criteria to complete sterilization for the target area surrounding the first UV sterilization unit.

The above and other preferred features, including various novel details of implementation and combination of events, will now be more particularly described with reference to the accompanying figures and pointed out in the claims. It will be understood that the particular systems and methods described herein are shown by way of illustration only and not as limitations. As will be understood by those skilled in the art, the principles and features described herein may be employed in various and numerous embodiments without departing from the scope of any of the present inventions. As can be appreciated from the foregoing and the following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

The foregoing Summary, including the description of some embodiments, motivations therefor, and/or advantages thereof, is intended to assist the reader in understanding the present disclosure, and does not in any way limit the scope of any of the claims.

### BRIEF DESCRIPTION OF THE DRAWINGS

The accompanying figures, which are included as part of the present specification, illustrate the presently preferred embodiments and together with the generally description given above and the detailed description of the preferred embodiments given below serve to explain and teach the principles described herein.

FIG. 1 illustrates, an automated UV sterilization system, in accordance with some embodiments.

FIG. 2 illustrates, an automated UV sterilization system having a plurality of UV units, in accordance with some embodiments.

FIG. 3 illustrates a flow chart for an automated UV sterilization process, in accordance with some embodiments.

FIG. 4 illustrates a multi-level UVC sterilization system, in accordance with some embodiments.

FIG. 5 illustrates a lower UVC unit of the multi-level UVC sterilization system, in accordance with some embodiments.

FIG. 6 illustrates a block diagram of an example computer system, in accordance with some embodiments.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

Apparatus and methods for automated UV sterilization are presented. It will be appreciated that, for simplicity and clarity of illustration, where considered appropriate, reference numerals may be repeated among the figures to indicate corresponding or analogous elements. In addition, numerous specific details are set forth in order to provide a thorough understanding of the exemplary embodiments described herein. However, it will be understood by those of ordinary skill in the art that the exemplary embodiments described herein may be practiced without these specific details.

As used herein, the UV sterilization systems will be described in the context of light emitting diodes (LEDs) emitting in the UVB and UVC spectrum. However, this is not limiting, and the configurations presented herein are applicable to other types of LEDs, including LEDs emitting in the UVA or visible spectrum. By way of example and not limitation, the automated UV sterilization system will be described in the context of a multi-level UV / UVC sterilization system. However, this is not limiting, and the configurations of the automated UV sterilization system described herein can include standalone UV sterilization systems, e.g., include UV sterilization systems that are of a single unit or that do not include a multi-level configuration.

In some examples, as described herein, the UV sterilization systems, UV sterilization processes and/or the UV sterilization products can include ultraviolet-C / UVC sterilization systems, UVC sterilization processes, and/or UVC sterilization products. In one example, as used herein, UV sterilization can include exposing an area and/or region to light having a wavelength of approximately within the UVB and/or UVC spectrum. In the UVC spectrum example, the UVC sterilization can include exposing an area at a wavelength of at least one of approximately 253.7 nm, or approximately 254 nm.

It can be important to control the cleaning and/or sterilization process performed by the UV sterilization systems, as UV exposure can be harmful to people and animals within a vicinity of the UV sterilization system while the UV sterilization system is in operation. In some examples, for safety, the UV sterilization process and/or products are generally used in unoccupied areas, e.g., regions and/or areas for cleaning that have been cleared of human and/or animals to prevent potential UV exposure. Thus, it can be useful to automate and control when and how long UV sterilization systems perform cleaning and/or sterilization processes.

To effectively clean and/or sterilize a target area, it can be important to determine where individual UV units of a UV sterilization system are placed within in a target area, the duration at which each UV unit performs cleaning and/or sterilization processes within the target area. In some examples, to effectively clean and/or sterilize the target area, UV sterilization products, e.g., such as UV Towers, are often placed within the target area, and are left for a particular amount of time to eliminate a target percentage of viruses, bacteria, and/or other hazardous micro-organisms from the target area. In a first example, it can take approximately 21-24 minutes to sterilize and/or cleanse an area and/or room using a 4-log sterilization rate (e.g., at approximately 99.99% sterilization), and part of the sterilization process can include taking approximately 9-10 minutes to pre-clean the room. Thus, there can be at least an 11-14 \-minute delay until the room and/or target area can be occupied and/or used. In a second example, other UV sterilization products can use tower lamps mounted at a height which may make them ineffective for sterilizing some hard to reach regions, e.g., the lamp height can be substantially elevated, and not configured to allow the for sterilizing and/or cleaning the hard to reach regions such as under beds, chairs, tables, etc. In a third example, UV sterilization products can include one or more UV lamps which can be configured to turn off the UV sterilization products at any time a human and/or animal is detected within the vicinity of the sterilization area, e.g., using sensors to detect the presence of the human/or animal. In a fourth example, the UV sterilization products can be configured to be movable, including a rolling mobile device that can be easily relocated from one location to another. In a particular example, the UV sterilization products can include smaller units that can be carried from place to place, where these smaller units may not be used for medium to large facilities and are usually utilized in homes, small offices and/or for vehicles such as ambulances. In a fifth example, to effectively use UV sterilization products, users may have to determine the size of each target area to be sanitized, as well as determine the appropriate power (e.g., in joules) needed to effectively clean a target area. In a sixth example, the user may have to determine how to control, activate and set an appropriate time and/or area to achieve a desired biological kill-rate and/or target sterilization for the given area. Provided these examples, there is an opportunity to improve the placement UV sterilization systems, and the duration of cleaning and/or sterilization performed by the UV sterilization systems, to improve overall the efficiency of the sterilization process within a given region, and maximize the sterilization process performed.

Furthermore, it can be difficult to determine if the UV sterilization process is completed, e.g., without appropriate sensing, feedback and due to potential interruptions in the sterilization process. In some examples, such a situation can require users to take additional time to verify if the sterilization process was interrupted, or whether the sterilization process had finished, by checking on the UV sterilization system itself in person. Provided the sterilization process was interrupted, the users may be required to leave the area, and return a later time to allow the sterilization process to continue and eventually complete. Thus, it can be beneficial to include a feedback mechanism to determine the amount of UV sterilization exposure for a given process, and to determine if any interruptions had occurred.

Also, other UV sterilization processes and products, e.g., such as conventional UV sterilization products and processes, may not be configured to optimally sterilize a target area. In some examples, the conventional UV sterilization products may not be configured to clean, and/or effectively sterilize, hard reach areas such as under beds, chairs, tables, etc. In the same example, the other UV sterilization products and process can take additional time to sterilize an area, which may force some users to leave the UV sterilization processes and product alone unattended during the sterilization process. Leaving the UV sterilization process and product alone can pose a risk to other users in case of malfunction while the UV sterilization product is unattended. Conventional UV sterilization systems may not include feedback mechanisms that provide information on whether a target amount of UV sterilization exposure had been reached, or that determine if any interruptions in the UV sterilization process had occurred. Conventional UV sterilization system may not adjust for losing UV radiation power over time, e.g., as the lifespan of a UV lamp (e.g., bulb) of the UV sterilization system diminishes.

Therefore, there is an opportunity to improve the efficiency and effectivity of UV sterilization systems and/or products to address the challenges described above.

In some embodiments, the UV sterilization systems described herein include UV LED devices having a set of UV LED circuits configured to produce and/or expose light at a target UV power output. In some examples, the UV sterilization systems can be configured to expose and/or provide a dosage of UV light to a target area. As described herein, the exposure and/or dosage of UV light produced by the UV sterilization system can be referred to as a UV dosage. The UV dosage can also be referred to herein as UVC exposure, UV exposure, UVC dosage, among other terms. Furthermore, the UV sterilization systems as described herein can be referred to as automated UV sterilization systems, UVC sterilization systems, UVC towers, UV towers, among other terms.

### Automated UV Sterilization System

One or more automated UV sterilization systems are presented herein which are configured to address the challenges of UV sterilization processes and/or products described above.

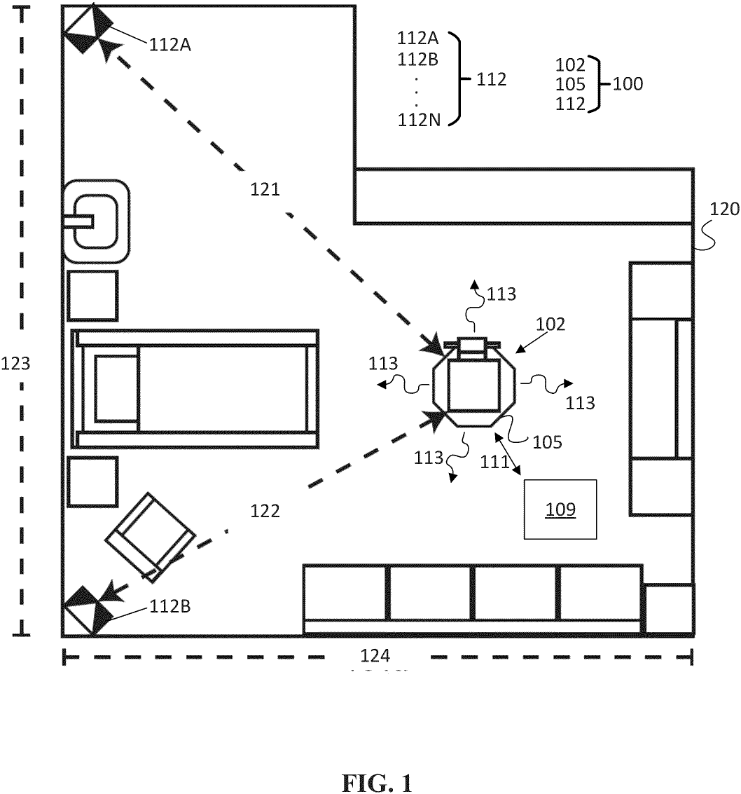

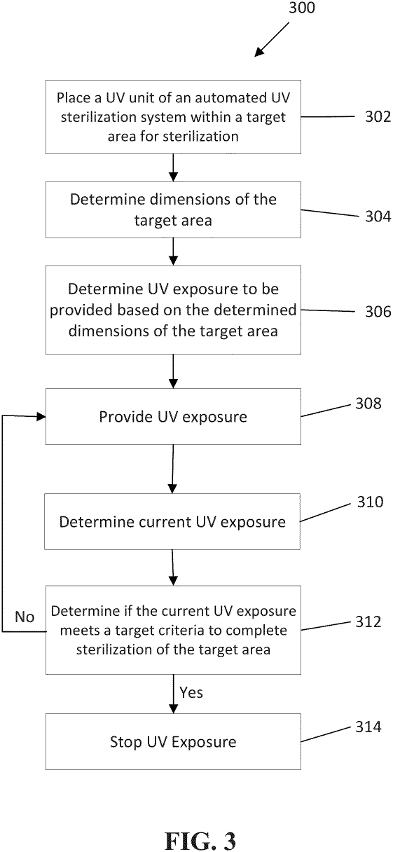

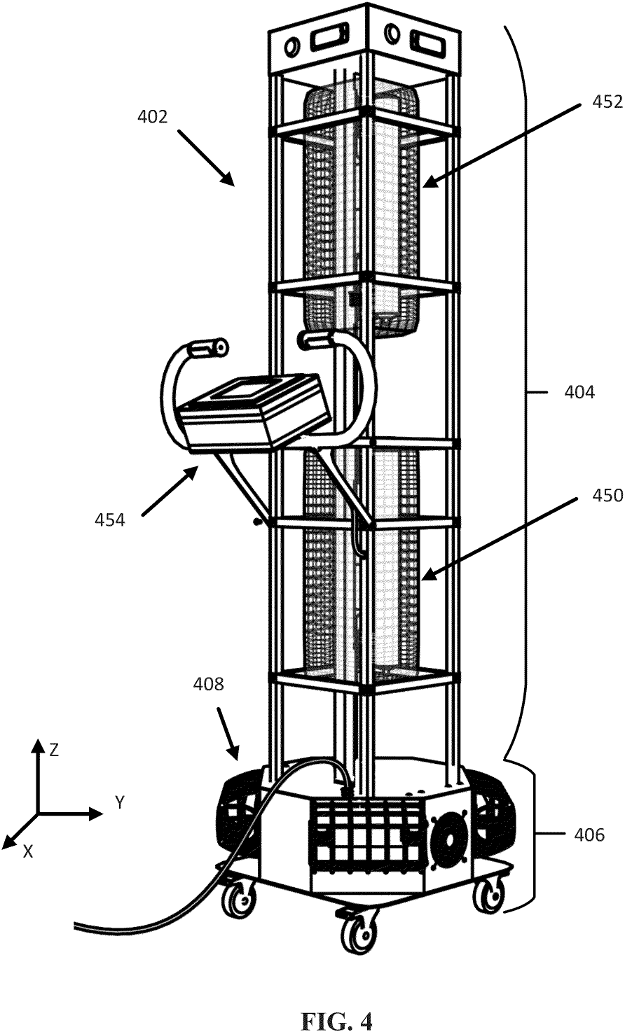

Referring to FIG. 1, an automated UV sterilization system is shown according to some embodiments. In some embodiments, the automated UV sterilization system 100 can include sensors 112A, 112B for detecting UV exposure, one or more UV units 102, e.g., UV sterilization units configured to provide UV exposure 113, among other systems and/or components. Although two sensors, 112A, 112B are shown, any number sensors, e.g., 1, 2, 3, etc., sensors can be used. In one example, up to N number of sensors, e.g., 1, 2, 3, ... N number of sensors 112N can be used, were N is an integer number. The sensors 112A, 112B for detecting the UV exposure can include dosimeters, power meters, among other sensors. The dosimeters can include electronics dosimeters, among other dosimeters. Collectively, the sensors 112A, 112B can be referred to as sensors 112. In some embodiments, the sensors 112 can be wirelessly connected to the UV units 102. The UV unit 102 can be configured to emit light having a wavelength of approximately within the UVB and/or UVC spectrum. Exemplary UV units 102 can include UV / UVC sterilization systems shown in FIGS. 3 and 4 and described below, among other UV sterilization devices. The automated UV sterilization system 100 can include wired sensors 105. In some examples, the wired sensors 105 can include power meters, dosimeters, among other sensors. The wired sensors 105 can include sensors connected directly to the UV unit 102 via a wired connection. As shown, the sensor 112A can be a first distance 121 away from the UV unit 102, and the second sensor 112B can be a second distance 122 away from the UV unit 102. A target area 120 for UV exposure can have a length 123 and a width 124. In a particular example, the target area can have a length 123 and width 124 of 16 ft. In some examples, the target area 120 can have an area of approximately 256 ft. In the same example, the first sensor 112A can be approximately 12 ft away from the UV unit 102, and the second sensor 112B can be approximately 10 ft away from the UV unit 102. The UV units 102 can also be referred to herein as UV sterilization units, among other terms.

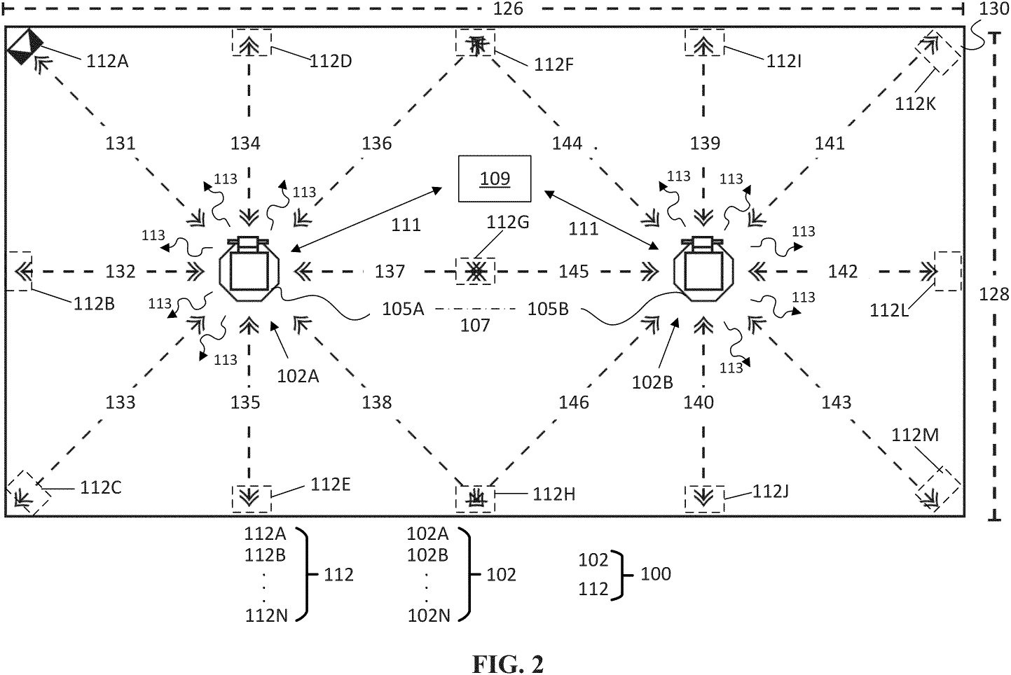

Referring to FIG. 2, an automated UV sterilization system having a plurality of UV units, is shown according to some embodiments. In some embodiments, the automated UV sterilization system 100 can include sensor 112A for detecting UV exposure, one or more UV units 102A, 102B, among other systems and/or components. The UV units can include a first UV unit 102A and a second UV unit 102B. Collectively, the UV units 102A, 102B can be referred to as UV units 102. Although two UV units 102A, 102B are shown, any number UV units, e.g., 102A, 102B ... 102N UV units can be used. In one example, up to N number of UV units, e.g., 1, 2, 3, ... N number of UV units 102N can be used, were N is an integer number. Although one sensor, 112A is shown, any number sensors, e.g., 112A, 112B, 112C, ... 112M sensors can be used, e.g., sensors 112B - 112N can be optional sensors.. In one example, up to N number of sensors, e.g., 1, 2, 3, ... N number of sensors 112N can be used, were N is an integer number. The sensors 112A - 112N for detecting the UV exposure can include dosimeters, power meters, among other sensors. Collectively, the sensors 112A - 112N can be referred to as sensors 112. In some embodiments, the sensors 112 can be wirelessly connected to the UV units 102A, 102B. As shown, a first sensor 112A can be a first distance 131 away from the first UV unit 102A, a second sensor 112B can be a second distance 132 away from the first UV unit 102A, a third sensor 112C can be a third distance 133 away from the first UV unit 102A, a fourth sensor 112D can be a fourth distance 134 away from the first UV unit 102A, a fifth sensor 112E can be a fifth distance 135 away from the first UV unit 102A, a sixth sensor 112F can be a sixth distance 136 away from the first UV unit 102A, a seventh sensor 112G can be a seventh distance 137 away from the first UV unit 102A, and an eighth sensor 112H can be an eighth distance 138 away from the first UV unit 102A. As shown, a ninth sensor 112I can be a ninth distance 139 away from the second UV unit 102B, a tenth sensor 112J can be a tenth distance 140 away from the second UV unit 102B, an eleventh sensor 112K can be an eleventh distance 141 away from the second UV unit 102B, a twelfth sensor 112L can be a twelfth distance 142 away from the second UV unit 102B, a thirteenth sensor 112M can be a thirteenth distance 143 away from the second UV unit 102B, the sixth sensor 112F can be a fourteenth distance 144 away from the second unit 102B, the seventh sensor 112G can be a fifteenth distance 145 away from the second UV unit 102B, and the eighth sensor 112H can be a sixteenth distance 146 away from the second UV unit 102B. In some examples, at least one of the sensors 112 can connect to one or more of the UV units 102. In one non-limiting example, the sixth sensor 112F, seventh sensor 112G and/or the eighth sensor 112H can connect to the first UV unit 102A and the second UV unit 102B, e.g., via a wireless connection and/or via a wired connection. A target area 130 for UV exposure can have a length 128 and a width 126. In a particular example, the target area 130 can have a length 128 of approximately 18 ft and width 126 of approximately 40 ft. In some examples, the target area 130 can have an area of approximately 720 ft. In the same example, the first distance 131, third distance 133, sixth distance 136, eighth distance 138, eleventh distance 141, thirteenth distance 143, fourteenth distance 144 and sixteenth distance 146 can be approximately 12 ft. In a particular example, the second distance 132, seventh distance 137, twelfth distance 142 and fifteenth distance 145 can be approximately 10 ft. In a particular example, the fourth distance 134, the fifth distance 135, the ninth distance 139 and the tenth distance 140 can be approximately 9 ft. The automated UV sterilization system 100 can include wired sensors 105A, 105B. In some examples, the wired sensors 105A, 105B can include power meters, dosimeters, among other sensors. The wired sensors can include a first wired sensor 105A and a second wired sensor 105B. The first wired sensor 105A can be connected to the first UV unit 102A and the second wired sensor 105B can be connected to the second UV unite 102B. The first and second wired sensors 105A, 105B can include sensors connected directly to the UV units 102A, 102B respectively, via a wired connection. In some examples, the first and second wired sensors 105A, 105B can optionally be connected together via a wired connection 107, e.g., allowing the wired sensors 105A, 105B to communicate to each other wired sensor via the wired connection 107.

Furthermore, although exemplary target areas 120, 130 are shown, any other target areas can be used. The target areas 120, 130 can include a target regions, rooms, and/or environments for sterilization and/or cleaning. Target areas 120, 130 can be referred to collectively as target area 150.

Referring to FIGS. 1 and 2, in some embodiment, the automated UV sterilization system 100 can include software and/or hardware configured to control the automated UV sterilization system, e.g., the automated UV sterilization system 100 can include control software and/or hardware. The control software and/or hardware can be configured to allow a user to control the automated UV sterilization system 100, e.g., directly via the UV units 102 and/or remotely using a remote control device 109. The remote control device 109 can include a laptop, a mobile phone, a tablet, among other electronic devices. In some examples, the automated UV sterilization system 100 can include wireless communication devices 109 configured to allow for the remote control and/or access of the automated UV sterilization system, e.g., via a wireless connection 111. The wireless connection 111 can include Wi-Fi, Bluetooth, infrared, among other remote communication methods and/or protocols. The automated UV sterilization system 100 can include wired and/or wireless security systems and/or protocols. The automated UV sterilization system 100 can be configured to protect and/or prevent an unauthorized user from accessing, using and/or controlling the automated UV sterilization system 100. As used herein, the wireless connection 111 can also be referred to herein as wireless communication.

Referring again to FIGS. 1 and 2, in some embodiments, the automated UV sterilization system 100 can be configured to automatically determine a target UV exposure 113 based on a target area for sterilization, e.g., the target areas 120, 130 surrounding one or more UV units 102 of the automated UV sterilization system 100 shown in FIGS. 1 and 2. The target area can include a target regions, rooms, and/or environments for sterilization and/or cleaning, e.g., the exemplary rooms in FIG. 1 and FIG. 2. In some examples, subsequent to placing UV units 102 within the target area, the automated UV sterilization system 100 can be configured to automatically determine the geometrical area, and/or volume of the target area, and based on the determined geometrical area and/or volume of the target area, calculate and expose the target area to a target UV exposure. The geometrical area and/or volume of a target area can also be referred to as a size of the target area. In one example, UV unit 102 of the automated UV sterilization 100 system can be placed at a middle and/or central location within a room (e.g., target areas 120, 130), detect and/or determine a size of the room, and based on the determined room size, calculate and/or determine UV exposure for that room. Once the UV exposure is determined, the automated UV sterilization system 100 can be initiated to provide the UV exposure.

Referring to FIGS. 1 and 2, in some embodiments, the automated UV sterilization system 100 can be activated directly, and/or remotely. In some examples, activating the automated UV sterilization system 100 directly can include initiating a button, switch and/or a control panel that is part of at least one UV unit of the UV sterilization system, e.g., in some examples, the control console 454 of FIG. 4. The automated UV sterilization system 100 can be activated remotely via a control device 109 and/or via software. The control device 109 can include a cellular phone, a smart phone, a tablet, a computer, a separate control device, and/or any other remote control device. The automated UV sterilization system 100 can be configured to be controlled via a wireless connection 111 and/or a wired connection. In some examples, the wireless connection 111 comprises a wireless connection via WiFi, Bluetooth, 3G/4G/5G, among other wireless communication protocols. In an non-limiting example, the automated UV sterilization system 100 can include wireless interface device (e.g., as part of the UV unit 102), such as an infrared device, WiFi card, Bluetooth dongle, ZigBee module, a 3G wireless modem, a 4G wireless modem, a 5G wireless modem, among other wireless devices. In some examples, wireless connections 111 can make use of an infrared communication protocol, 2.4 ghz frequency wireless connection, a 5.8 ghz wireless connection, a 3G/4G/5G network communication, among other wireless connections and/or communication protocols. In a non-limiting example, the control device can be directly connected to the UV unit 102 via a wired connection, e.g., via an Ethernet connection, RS-232 cable, among other wired connections. In one particular non-limiting example, the control device can also include sensors 105A, 10B, and can be connected via the wired connections shown for the wired sensors 105A, 105B, e.g., to initiate the UV exposure from the UV unit 102. Thus the control device, connected via a wireless connection and/or a wired connection, can be used to initiate the UV exposure from the UV unit 102.

Referring to FIGS. 1 and 2, in some embodiments, the automated UV sterilization system 100 can automatically control the UV exposure 113 based on input from one or more sensors 112. The sensors 112 can include a power meter, a dosimeter, among other sensors and/or devices. In some examples, the UV units 102 of the automated UV sterilization system 100 can be configured to receive power consumption information from a power meter, UV exposure information from a dosimeter, among other sensor input. The UV units 102 can be configured to control the UV exposure of the automated UV sterilization system 100 based on the received sensor input. The automated UV sterilization system 100 can be configured to control the UV exposure based on the received sensor input over a duration of time. In one example, the automated UV sterilization system 100 can receive power consumption information from the power meter and/or UV exposure readings from the dosimeter over a duration of time, and based on the received power consumption and UV exposure readings over the duration of time, determine subsequent UV exposure to cleanse and/or sterilize a target area (e.g., the target areas 120, 130). Provided the automated UV sterilization system 100 detects decreasing power draw and/or decreasing UV exposure over time, the automated UV sterilization system 100 can control and/or adjust the UV exposure and extend the UV exposure duration to meet a particular UV exposure target. Once a target power consumption and/or UV exposure reading has been reached, the automated UV sterilization system 100 can be configured to stop further UV exposure. The UV units 102 can be connected to the power meter and/or dosimeter via the wired and/or wireless connection. In some embodiments, each of the sensors 112 can communicate with each other sensor of the sensors 112 via a wired and/or wireless connection, e.g., via one of the wired and/or wireless connections described herein.

Referring again to FIGS. 1 and 2, in some embodiments, the automated UV sterilization system 100 can be configured to be autonomous and/or independent. The automated UV sterilization system 100 can be configured to calculate the appropriate UV exposure of the target area (e.g., the target areas 120, 130) without prior input, and/or training from a user, e.g., prior to allowing the automated UV sterilization system 100 to determine the room size and activating the UV exposure. The automated UV sterilization system 100 can be configured to calculate a target power, e.g., in joules over time, to be used to generate the target UV exposure based on the room size of the target area.

Referring to FIGS. 1 and 2, in some embodiments, the automated UV sterilization system 100 can be configured to determine an the UV exposure 113 for the target area based on a lifespan and/or depreciation of a UV lamp (e.g., bulb) used by the UV units 102 of the automated US sterilization system 100. In some examples, the automated UV sterilization system 100 can be configured to determine an exposure duration for the UV exposure 113 based on the life span of the UV lamp and/or the size of the target area. In one example, the automated UV sterilization system 100 can be configured to determine how much more time to extend the UV exposure 113 provided the life span of the UV lamp used is halfway, three-fourths, etc. closer to the end of life of the UV lamp used. In one non-limiting example, it can take a particular duration to sterilize a target area at the beginning of a UV lamp’s life span, and that it can take up to 2 to 3 times longer than the original duration to sterilize the same target area as the UV lamp nears its end of life. In some examples, the automated UV sterilization system 100 can be configured to compensate by extending the UV exposure 113 time by 2 to 3 times longer, or as necessary, to attain a target cleanse and/or sterilization of the target area. In a particular non-limiting example, provided the automated UV sterilization system 100 can take approximately 4.5 minutes to attain a target UVC power output (measured in joules) to sterilize a target area for a UV lamp at the beginning of the UV lamp’s life span, and the same UV sterilization system can take approximately 10-12 minutes to achieve the same UVC power output for the UV lamp sometime near the end of the UV lamp’s life span, the UV sterilization system can be configured to perform UV exposure of the target area at an extended duration, e.g., approximately between 10-12 minutes, to achieve the same UVC power output for the UV lamp near the end of the UV lamp’s life span.

### Methods for Automated UVC Sterilization System

FIG. 3 is a flowchart of an example method 300 for an example automated UV sterilization process. In step 302, a user can place a UV unit of an automated UV sterilization system within a target area for sterilization. The user can place the UV unit at a middle and/or central location of the target area. In a step 304, the automated UV sterilization system can determine the dimensions of the target area. In some examples, the automated UV sterilization system can use sensors and/or software to determine an area, volume and/or size of the target area, where the dimensions of the target area can include the area, volume and/or size of the target area. In step 306, the automated UV sterilization system can determine the UV exposure to be provided for sterilizing the target area based on the determined dimensions of the target area. In step 308, the automated UV sterilization system provides the UV exposure. In a non-limiting example, the automated UV sterilization system can activate the UV unit to provide the UV exposure. In a non-limiting example, the automated UV sterilization system can activate the UV exposure provided by one or more UV sterilization units. In some examples, the automated UV sterilization system first performs a safety check to determine if a human and/or animal is present, and if there is nothing detected, the automated UV sterilization system engages the UV exposure. In step 310, the automated UV sterilization system can determine a current UV exposure for the target area. In some examples, the automated UV sterilization system can use sensors to determine a current UV exposure for the target area. The sensors can include a dosimeter and/or a power meter. In a step 312, the automated UV sterilization system can determine if the current UV exposure meets a target to complete sterilization of the target area. The automated UV sterilization system can determine if the current UV exposure meets a target criteria to complete sterilization of the target area. The target criteria can be based on the dimension of the target area, the UV exposure dosage already provided over time, a life span of a UV lamp used by the automated UV sterilization system, among other factors. If the current UV exposure does not meet the target criteria to complete sterilization, the method proceeds back to step 308. If the current UV exposure meets the target criteria to complete sterilization, the method proceeds to step 314. In step 314, the automated UV sterilization system stops the UV exposure.

Exemplary UV sterilization systems and/or UV sterilization units that can be used with the automated UV sterilization system described above, are presented below.

### Multi-Level UVC Sterilization System

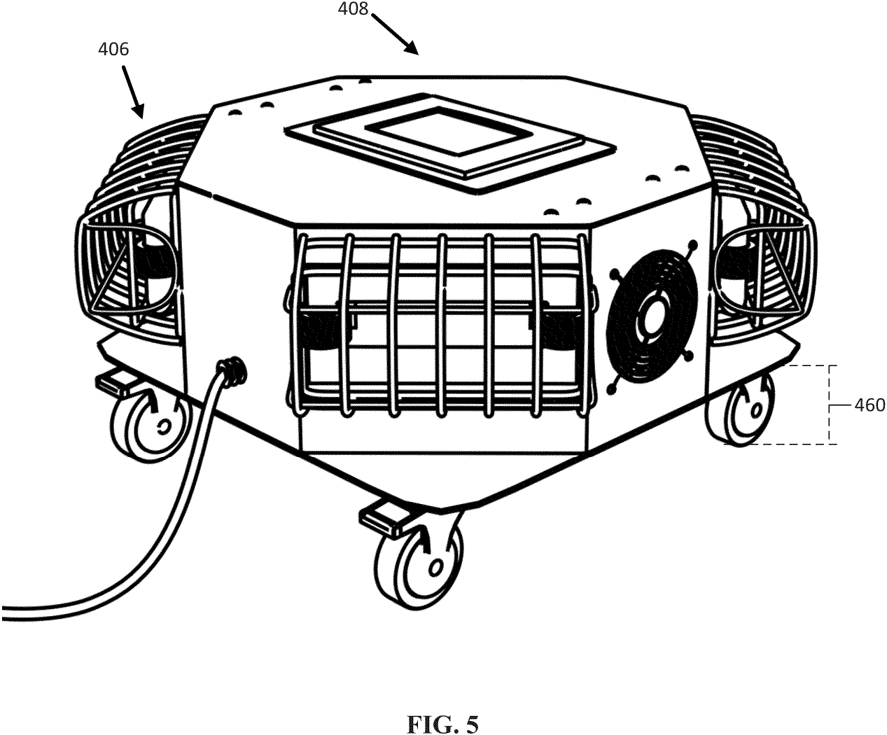

One or more multi-level UVC sterilization systems are presented herein which are configured to address the challenges of UV sterilization and/or UVC sterilization processes and/or products described above. As used herein, the multi-level UVC sterilization system can also be referred to as a multi-level UVC tower, a UVC tower, a tower, among other terms. In a first example, in place of and/or in addition to using standard fluorescent/quartz type UVC lamps, the multi-level UVC sterilization system can be configured to use one or more high intensity UVC induction lamps. Although one embodiment can include the multi-level UVC sterilization system using high intensity UVC induction lamps, in another embodiment, the multi-level UVC sterilization system can use standard fluorescent/quartz type UVC lamps. The UVC induction lamps can be configured to decrease the sterilization time for sterilizing a target area. The decrease in sterilization time can include, in one example, decreasing the sterilization time to approximately under 10 minutes which can eliminate and/or substantially reduce wait time to finish a sterilization process for a designated target area. In a second example, the multi-level UVC sterilization system can include one or more UVC lamps mounted just approximately above ground level (e.g., referring to Height 460 shown in FIG. 5). The Height 460 can include a height of approximately 6-12 inches. The multi-level UVC sterilization system can be configured to sterilize, clean, and/or cleanse hard to reach areas, such as under furnishings, under beds, chairs, tables, etc. In a third example, the multi-level UVC sterilization system can include strobe warning lights. As used herein, the strobe warning lights can also be referred to as strobe lights, warning lights, among other terms.

In some embodiments, the strobe warning lights can be included on one side, or all sides, of the multi-level UVC sterilization system. The multi-level UVC sterilization system can include software and/or hardware configured to allow programming of the strobe warning lights. The software and/or hardware can be configured to allow the strobe warning lights to be programmed when the multi-level UVC sterilization system is not in use and/or is interrupted, e.g., while not in operation and/or after the cleansing process has been completed. In an example, the strobe lights be configured to allow users to be informed if and/or when the multi-level UVC sterilization system has been interrupted. Such a configuration can also allow users to be informed when the multi-level UVC sterilization system has a sterilization processes which needs to be finished, or if and/or when the sterilization process is completed (e.g., saving sterilization time and labor on the user’s part to check a system’s status). The strobe warning lights can be configured to save sterilization time by allowing users to be informed if and/or when the multi-level UVC sterilization system status without the user having to enter the area under sterilization and/or that is being cleansed.

Referring to FIG. 4, an exemplary multi-level UVC sterilization system is shown, according to some embodiments. The multi-level UVC sterilization system 402 can include a 2-piece, e.g., multi-level, UVC unit configuration. The multi-level UVC sterilization system 402 can include a first UVC unit 404 and a second UVC unit 406. The first UVC unit can be referred to as an upper UVC unit 404, and the second UVC unit can be referred to as a lower UVC unit 406. In some examples, the upper and lower UVC units 404, 406, can include one or more UVC induction lamps and/or UV lamps 408, 450, 452. As shown the upper UVC unit 404 can include one or more lamps 450, 452 positioned along a vertical Z-axis. In one example as shown in FIG. 4, the first and second upper UVC lamps 450, 452 are positioned along the z-axis. The lower UVC unit 406 can be configured to be used as a base for the multi-level UVC sterilization system 402, e.g., the upper UVC unit 404 can be configured to be positioned over and/or on top of the lower UVC unit 406. The lower UVC unit 406 can be configured to be used as a separate UVC system alone for lower elevation sterilization and/or cleansing. The lower UVC unit 406 can include one or more UVC induction lamps. As shown, in one example, the lower UVC unit 406 can include a first lower UVC lamp 408. As described herein, a UVC unit can be referred to as a UV unit, among other terms.

In some embodiments, the multi-level UVC sterilization system can be configured to inhibit UVC shadowing. In some examples, UVC shadowing can include regions of reduced illumination on the target area of interest, due to obstructions in a light path between the UVC unit and the target area of interest. In one example, using a single UVC unit to illuminate a target area can limit the total illumination that is received by the target area, e.g., due to potential obstructions between the UVC unit and the target area, where in contrast multiple UVC units can more uniformly illuminate an area by illuminating under, over and/or around such obstructions. Providing substantial increased light exposure to illuminate through the obstructions using multiple UVC units can provide improved sterilization, cleansing, cleaning and/or disinfection due to the additive exposure to UVC light. Furthermore, by utilizing UVC units in a stacked configuration, as shown in FIG. 4 having a tower configuration using multiple UVC lamps, the UVC sterilization system can further provide UVC light illumination under, over and/or around obstructions to provide improved exposure to a target area in comparison to a UVC system using a single UVC unit. Also, a UVC unit can be configured to use short UVC waves of light, and thus, provided short wave photons can be minimally deflected and reflected, the stacked UVC unit configuration can further inhibit UVC shadowing as compared to other UV sterilization products or systems.

Although a 2-piece UVC sterilization system is shown in FIG. 4, any number of UVC unit configuration can be used. In some examples, 3-piece, 4-piece, or more pieces or UVC units can be used.

Referring again to FIG. 4, the multi-level UVC sterilization system 402 can include a control console 454 configured to control the multi-level UVC sterilization system 402. In some examples, the control console 454 can be configured to allow one or more users to control multi-level UVC sterilization system. The control console 454 can be configured to allow a user to turn the system on, turn the system off, the control the UVC exposure for the entire system, the control the UVC exposure UVC unit, e.g., the upper and lower UVC units, to control the UVC exposure per UVC lamp, among other UVC system controls. The control console 454 can include safety and/or security systems and/or protocols configured to prevent unauthorized users from accessing and/or controlling the multi-level UVC sterilization system 402. In one example, the control console 454 can request a user for credentials and/or verification prior to allowing access and/or control to the multi-level UVC sterilization 402.

In some embodiment, the multi-level UVC sterilization system can include software and/or hardware configured to control the multi-level UVC sterilization system, e.g., the multi-level UVC sterilization system can include UVC control software and/or hardware. The UVC control software and/or hardware can be configured to allow a user to control the multi-level UVC sterilization system, e.g., directly via the control console and/or remotely using a remote control device. The remote control device can include a laptop, a mobile phone, a tablet, among other electronic devices. In some examples, the multi-level UVC sterilization system can include wireless communication devices configured to allow for the remote control and/or access of the multi-level UVC sterilization system. The wireless communication devices can include Wi-Fi, Bluetooth, infrared, among other remote communication methods and/or protocols. The multi-level UVC sterilization system can include wired and/or wireless security systems and/or protocols. The multi-level UVC sterilization system can be configured to protect and/or prevent an unauthorized user from accessing, using and/or controlling the multi-level UVC sterilization system.

Referring to FIG. 5, the lower UVC unit of the multi-level UVC sterilization system is shown, according to some embodiments. In some examples, the lower UVC unit 406 can be configured to be used in hard to reach areas, such as under beds, tables, etc., and/or used alone (e.g., independent of the multi-level UVC sterilization system of FIG. 4) such as in restaurants, hospitals, etc. In some examples, the lower UVC unit 406 can be configured to allow sterilization, cleaning, cleansing and/or manual bleaching under furnishings such as beds, tables, chair, etc. and anywhere the lower UVC unit 406 can be placed. As shown, the lower UVC unit 406 can include a first lower UVC lamp 408.

In some embodiments, the multi-level UVC sterilization system can include a UVC exposure control system. As referred to herein, the UVC control system can also be referred to as a UVC exposure control system. In some examples, the UVC control system can be configured to determine the exposure and/or dosage of UVC light for a target area. The UVC control system can be configured to control the upper and lower UVC units, e.g., to determine and/or control a target UVC exposure for the target area. The UVC control system can include a UVC dosimeter. The UVC dosimeter can be configured to determine the UVC exposure and/or dosage of the target area. In one example, the UVC control system can be configured to control the UVC exposure to provide for an approximately 15 minute sterilization, cleansing and/or cleaning duration. The UVC control system can be configured to provide for a Log4 efficacy. The UVC control system can be configured to determine a target UVC exposure as based on UVC lamp life, number of UVC lamps, positioning of the UVC lamps, the height of UVC lamps, and/or other UVC exposure factors. In one example, the UVC control system can be configured to increase and/or decrease the duration of UVC exposure for the target area based on one or more of the UVC exposure factors.

In some embodiments, the multi-level UVC sterilization system can include on-board batteries and/or an on-board battery charger. In some examples, the on-board batteries and/or charger can be configured to allow for a 2,500 recharge cycle. The on-board batteries can include lithium ion batteries, among other types of batteries. The on-board batteries can be replaceable. The multi-level UVC sterilization system can include a power management system. The power management system can be configured to maximize the useful life of the on-board batteries and/or an on-board battery charger based on the UVC unit and/or UVC lamp usage.

### Hardware and Software Implementations

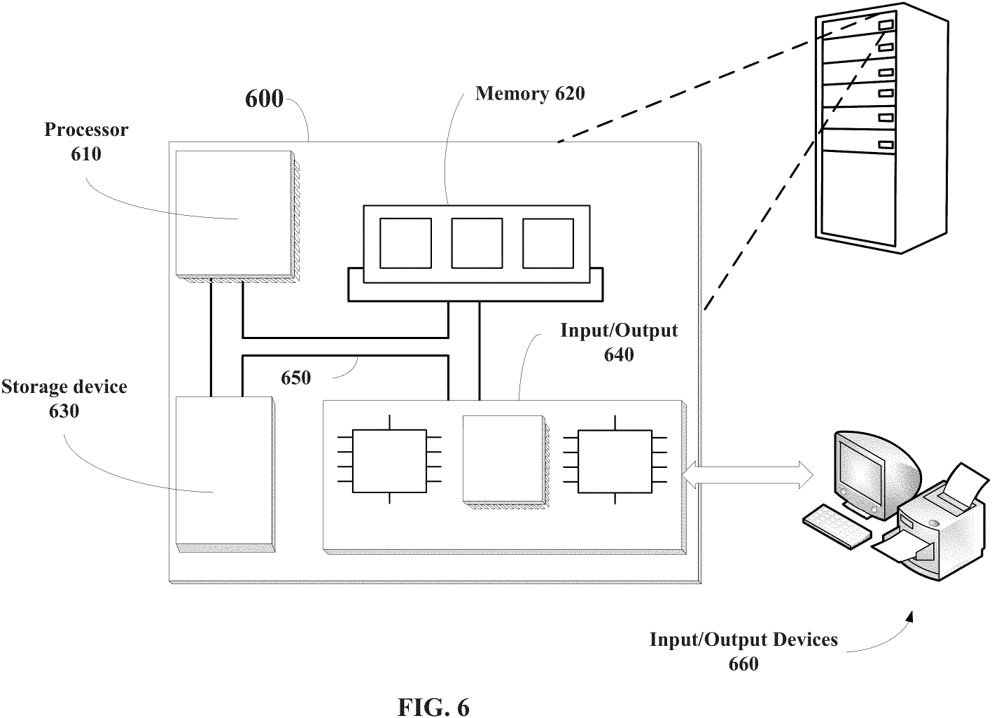

FIG. 6 is a block diagram of an example computer system 600 that may be used in implementing the technology described in this document. General-purpose computers, network appliances, mobile devices, or other electronic systems may also include at least portions of the system 600. The system 600 includes a processor 610, a memory 620, a storage device 630, and an input/output device 640. Each of the components 610, 620, 630, and 640 may be interconnected, for example, using a system bus 650. The processor 610 is capable of processing instructions for execution within the system 600. In some implementations, the processor 610 is a single-threaded processor. In some implementations, the processor 610 is a multi-threaded processor. The processor 610 is capable of processing instructions stored in the memory 620 or on the storage device 630.

The memory 620 stores information within the system 600. In some implementations, the memory 620 is a non-transitory computer-readable medium. In some implementations, the memory 620 is a volatile memory unit. In some implementations, the memory 620 is a non-volatile memory unit.

The storage device 630 is capable of providing mass storage for the system 600. In some implementations, the storage device 630 is a non-transitory computer-readable medium. In various different implementations, the storage device 630 may include, for example, a hard disk device, an optical disk device, a solid-date drive, a flash drive, or some other large capacity storage device. For example, the storage device may store long-term data (e.g., database data, file system data, etc.). The input/output device 640 provides input/output operations for the system 600. In some implementations, the input/output device 640 may include one or more of a network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 660. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 630 may be implemented in a distributed way over a network, for example as a server farm or a set of widely distributed servers, or may be implemented in a single computing device.

Although an example processing system has been described in FIG. 6, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; and magneto optical disks. The processor and the memory can be supplemented by, or incorporated in, special purpose logic circuitry.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An automated UV sterilization system, comprising:
a first UV sterilization unit comprising a first UV lamp, the first UV sterilization unit configured to provide UV exposure;
a first sensor coupled to the first UV sterilization unit, the first sensor configured to measure the UV exposure; and
the automated UV sterilization system configured to:
determine dimensions of a target area surrounding UV sterilization units;
determine the UV exposure to be provided based on the determined dimensions;
activate the UV exposure to be provided by UV sterilization units;
determine a current UV exposure within the target area; and
determine whether the current UV exposure meets a target criteria to complete sterilization for the target area surrounding UV sterilization units.

2. The automated UV sterilization system of claim 1, further comprising a second UV sterilization unit comprising a second UV lamp, the second UV sterilization unit configured to provide UV exposure.

3. The automated UV sterilization system of claim 1, wherein the first sensor is wirelessly coupled to the first UV sterilization unit.

4. The automated UV sterilization system of claim 1, wherein the first sensor comprises at least one of a dosimeter or a power meter.

5. The automated UV sterilization system of claim 1, further comprising a second sensor coupled to the first UV sterilization unit, the second sensor configured to measure UV exposure.

6. The automated UV sterilization system of claim 2, further comprising a second sensor coupled to the first and second UV sterilization units, the second sensor configured to measure UV exposure.

7. The automated UV sterilization system of claim 1, wherein the dimensions comprise at least one of an area or a volume of the target area.

8. The automated UV sterilization system of claim 1, wherein determining the current UV exposure comprises detecting the UV exposure using input from the first sensor.

9. The automated UV sterilization system of claim 1, wherein the input from the first sensor comprises input from a dosimeter.

10. The automated UV sterilization system of claim 1, wherein the input from the first sensor comprises input from a power meter.

11. The automated UV sterilization system of claim 1, wherein determining the current UV exposure comprises detecting the UV exposure over a duration of time.

12. The automated UV sterilization system of claim 1, wherein determining the current UV exposure comprises detecting the power consumption of the UV lamp.

13. The automated UV sterilization system of claim 1, wherein the target criteria is based on the dimensions of the target area.

14. The automated UV sterilization system of claim 1, wherein the target criteria is based on a life span of the UV lamp.

15. A method, comprising:
determining dimensions of a target area surrounding a first UV sterilization unit of an automated UV sterilization system, the first UV sterilization unit configured to provide UV exposure;
determining the UV exposure to be provided based on the determined dimensions;
activating the UV sterilization system to provide UV exposure;
determining a current UV exposure within the target area; and
determining whether the current UV exposure meets a target criteria to complete sterilization for the target area surrounding the first UV sterilization unit.

16. The method of claim 15, wherein the dimensions comprise at least one of an area or a volume of the target area.

17. The method of claim 15, wherein determining the current UV exposure comprises detecting the UV exposure using input from the first sensor.

18. The method of claim 15, wherein the input from the first sensor comprises input from a dosimeter.

19. The method of claim 15, wherein the input from the first sensor comprises input from a power meter.

20. The method of claim 15, wherein determining the current UV exposure comprises detecting the UV exposure over a duration of time.

21. The method of claim 15, wherein determining the current UV exposure comprises detecting the power consumption of the UV lamp.

22. The method of claim 15, wherein the target criteria is based on the dimensions of the target area.

23. The method of claim 15, wherein the target criteria is based on a life span of the UV lamp.
